(19) United States
(12) Patent Application Publication
(10) Pub. No.: US 2009/0175833 A1
(43) Pub. Date: Jul. 9, 2009

(54) PERICYTES FOR USE AS STEM CELLS

(76) Inventors: Paula Dore-Duffy, Detroit, MI (US); Andre Katyshev, Detroit, MI (US); Xueqian Wang, Detroit, MI (US)

Correspondence Address: KOHN & ASSOCIATES, PLLC, 30500 NORTHWESTERN HWY, SUITE 410, FARMINGTON HILLS, MI, 48334, US

(21) Appl. No.: 12/160,860

(22) PCT Filed: Jan. 16, 2007
(86) PCT No.: PCT/US07/01107

§ 371 Date: Nov. 17, 2008

Related U.S. Application Data
(60) Provisional application No. 60/759,258, filed on Jan. 13, 2006.

(51) Int. Cl. C12N 5/06; A61K 35/12; C12Q 1/02

(52) U.S. Cl. 424/93.7; 435/377; 435/325; 435/29

(57) ABSTRACT

A method of promoting perictye differentiation by selectively culturing pericytes in an enriched environment containing a promoter specific to a type of differentiation. Isolated and purified multipotent pericytes or pericyte precursors are provided. A stem cell therapy replacement comprising isolated and purified multipotent pericytes. A treatment of disease comprising an effective amount of isolated and purified multipotent pericytes or pericyte precursors is provided.

### BACKGROUND OF THE INVENTION

1. Technical Field

The present invention relates to pericytes. More specifically, the present invention relates to the use of pericytes as stem cells.

2. Description of the Related Art

The presence of adult multipotent “stem” cells has been demonstrated in a large number of tissues, for example the bone marrow, blood, liver, muscle, the nervous system, and in adipose tissue. Adult “stem” cells, which in theory are capable of infinite self-renewal, have great cell plasticity, i.e. the ability to differentiate into tissues other than those for which it was believed they were destined. The properties of the cells, which are similar to those of embryonic stem cells (ES), open up considerable therapeutic possibilities especially as the use does not pose the problems of compatibility and ethics, encountered with ES cells.

Unfortunately, medical application of the adult stem cells is currently extremely limited for two main reasons. First, it is very difficult to isolate the cells. Stem cells are very rare in an organism and very little is currently known about them, in particular at a molecular level, rendering direct purification impossible. There is a method for enriching multipotent cells, a population known as side population (“SP”), based on the capacity to exclude a vital stain (Goodell M A et al. (1996), J Exp Med. vol 83, 1797-1806; Zhou S et al. (2001) Nature Medicine, vol 7, 1028-1034). Other methods involve positive or negative selection, based on the presence or absence of cell markers. As an example, International Patent Application WO 01/11011 describes the depletion of bone marrow cells of CD45+ glycophorin A+ cells followed by culturing CD45−/GlyA− cells in the presence of growth factors. A similar method has been described by Reyes et al (Blood, November 2001, vol. 98, no 9, 2615-2625). Second, prior amplification of the cells in the undifferentiated state in vitro poses a major problem.

The existence of multipotent stem cells within the adult brain has been a topic of considerable research emphasis and the subject of some debate (McKay, 1997; Temple, 2001). The identity of this quiescent totipotent stem cell is still unknown. However, it is clear that stem cells tend to reside in specialized tissue (stem cell niche) and that the local microenvironment regulates their differentiation and self-renewal (Sprading et al, 2001; Doetsch, 2003). Stem cell niches appear to be relatively compartmentalized areas composed of a basement membrane, extracellular matrix, and cells thought to provide important signaling molecules that enhance and support neurogenesis (Lim and Alvarez-Buylla, 1999; Song et al, 2002). In the brain, two major germinal regions have been characterized (McKay, 1997; Temple, 2001). These are the subventricular zone (SVZ) and the subgranular zone (SGZ) (Sprading et al, 2001). Both the SVZ niche and the SGZ niche are in close association with the microvasculature, as well as perivascular cells, a specialized basal lamina and an extracellular matrix bed (Doetsch, 2003; Palmer et al, 2000). These elements are common components of the microvasculature and underscore their importance to the SVZ and SGZ germinal centers. The vascular niche also provides ample opportunity for extensive cell-cell communication (Palmer et al, 2000). The role of the microvasculature in central nervous system(CNS) stem cell differentiation is, however, still unclear.

The CNS microvascular endothelium is composed of two cellular constituents, the endothelial cell (EC) and the pericyte. Both cells are in close association with astrocytes, the basal lamina as well as numerous matrix proteins (Ballabh et al, 2004). In fact, the vascular pericyte synthesizes most elements of the basal lamina including a number of proteoglycans (Dienfenderfer and Brighton, 2000; Ozerdem et al, 2002). Pericyte synthesis and release of laminal proteins is thought to be a critical step in the differentiation of the blood-brain barrier (Korn et al, 2002).

Microvascular perivascular cells (“pericytes”) are defined by their location in vivo. The pericyte is a small ovoid shaped cell with many finger-like projections that parallel the capillary axis and partially surround an endothelial cell in a microvessel. Pericytes share a common basement membrane with the endothelial cell. Pericytes are elongated cells with the power of contraction that have been observed to have a variety of functional characteristics. Pericytes are widely distributed in the body and include mesangial cells (in the glomeruli of the kidney), Rouget cells, or mural cells (in the retina of the eye) (Hirschi & D'Amore, Cardiovasc Res October 1996; 32 (4):687-98.). Some of the pericyte functional characteristics observed in vivo and in vitro are that they regulate endothelial cell proliferation and differentiation, contract in a manner that either exacerbates or reduces endothelial cell junction inflammatory leakage, synthesize and secrete a wide variety of vasoactive autoregulating agonists, and synthesize and release structural constituents of the basement membrane and extracellular matrix (Shepro et al, FASEB J August 1993; 7 (11):1031-8.) Pericytes have thus been implicated as playing a role in vasoconstriction as well as a role in capillary blood flow, in the formation of blood vessels, in the immune response (particularly in the central nervous system), and in the extrinsic coagulation pathway.

Pericytes are thought to be derived from undifferentiated mesenchymal cells that are recruited by primordial endothelium and then differentiate into pericytes in microvessels or smooth muscle cells in large vessels. Pericytes are also pluripotent progenitor cells and have been shown to differentiate into a variety of different cell types, including osteoblasts, chondrocytes, adipocytes, phagocytes, fibroblasts, and smooth muscle cells. (Sims, 2000, Clin. Exp. Ped. Physiol., 27:842-846.) Pericytes behave in a manner similar to osteoblasts in vitro, by forming a mineralized extracellular matrix and expressing a number of genes that are also expressed by osteoblasts. These cells also form a well-defined matrix of bone, cartilage, and fibrous tissue in vivo. (Doherty and Canfield, Crit Rev Eukaryot Gene Expr 9 (1):1-17, 1999; Hirschi et al., Cardiovasc Res October; 32 (4):687-98, 1996.)

Pericytes have been identified in the inner intimae, the outer media, and in the vasa vasora of the adventitia of large, medium and small human blood vessels. Pericytes are associated with capillaries and post-capillary vessels. In fact, there are pericytes in all tissues containing capillaries Recent studies have suggested that pericytes in the arteries may be responsible, at least in part, for mediating the vascular calcification commonly associated with atherosclerosis (Canfield et al., Z Kardiol 2000; 89 Suppl 2:20-7.) Myxomatous tissue is a characteristic component of human coronary artery lesions and is found more often in restenotic lesions. This tissue represents a bulky accumulation of stellate-shaped cells of unknown histogenesis that are embedded in a loose stroma and may be involved in an immune response. Stellate cells (liver pericytes) represented a heterogeneous population, sharing features of smooth muscle cells (SMCs), macrophages, as well as antigen-presenting dendritic cells. Some workers have concluded that stellate cells of myxomatous tissue represent a specific phenotype of mesenchymal cells, possibly pericytes, which is activated to express some markers of antigen-presenting cells. (Tjurmin et al., Arterioscler Thromb Vasc Biol January 1999; 19 (1):83-97.)

Previous studies have shown that pericytes can differentiate into various cell types such as adipocytes, chondrocytes, fibroblasts and macrophages (Canfield et al., 2000; Diaz-Flores et al., 1992). These cells may therefore reflect in some aspects the phenotype of mesenchymal stem cells (MSC) originally isolated from bone marrow stroma (Caplan, 1991).

Pericytes or cells with probable pericyte-like morphology have been reported to differentiate to osteogenic or osteoblast/like cells in vitro (Canfield et al, 1996; Reilly et al., 1998). In serum-containing culture medium, CNS pericytes (3- to 7-day-old) take on a macrophage/dendridic cell phenotype (Balabanov et al, 1996, 1999) and then ultimately continue differentiating to form nodules that produce mineralized bone (Canfield et al, 1996, Dore-Duffy, unpublished observations). These data support the concept that CNS pericytes may be mesenchymal stem cells. Still additional evidence suggests that pericytes may differentiate to fibroblasts (Doherty and Canfield, 1999; Gerhardt and Betsholtz, 2003), ECs (Chaudhry et al, 1978), adipocytes (Cinti et al, 1984), chondrocytes (Reilly et al, 1998), and macrophages/dendritic cells (Balabanov et al, 1996, 1999).

It has been reported that the growth factors basic fibroblast growth factor (bFGF) and epidermal growth factor (EGF) stimulate pericyte proliferation and angiogenesis (Nico et al, 2004). Both EGF and bFGF-responsive vascular stem cells have been reported in the rat microvasculature (Palmer et al, 2000; Jin et al, 2002; Louissant et al, 2002). However, direct demonstration of a precursor cell displaying stem cell characteristics has not been forthcoming.

It would therefore be useful to develop a method of utilizing pericytes cell replacements for treating disease. The use would create a new source of therapeutics derived from the pericytes without much of the controversy or problems associated with adult and fetal stem cells.

### SUMMARY OF THE INVENTION

According to the present invention, there is provided a method of promoting perictye differentiation by selectively culturing pericytes in an enriched environment containing a promoter specific to a type of differentiation. Isolated and purified multipotent pericytes or pericyte precursors are provided. A stem cell therapy replacement comprising isolated and purified multipotent pericytes is also provided. A treatment of disease comprising an effective amount of isolated and purified multipotent pericytes or pericyte precursors is provided.

### BRIEF DESCRIPTION ON THE DRAWINGS

Other advantages of the present invention will be readily appreciated as the same becomes better understood by reference to the following detailed description when considered in connection with the accompanying drawings wherein:

FIGS. 1A through 1F are graphs showing the purity of freshly isolated CNS capillary suspensions;

FIGS. 2A through 2C and 2E through 2F are photographs showing the characterization of nestin/NG2-positive cells in CNS capillaries; FIG. 2D is a graph depicting the results;

FIGS. 3A and 3B are photographs of the results of RT-PCR showing the expression of nestin and NG2 transcripts in cultured CNS primary pericytes, FIGS. 3C and 3D are graphs depicting the statistical results;

FIGS. 4A through 4E are photographs showing that primary CNS pericytes are responsive to bFGF;

FIGS. 5A through 5R are photographs showing the characterization of differentiated pericyte-derived spheres;

FIGS. 6A through 6H are photographs showing the antigenic properties of cells cultured from pericyte derived spheres; and

FIG. 7 is a flow diagram depicting the antigenic properties of CNS capillaries and primary pericytes, wherein the sequence represents the isolation and culture of primary pericytes in serum containing medium and pericyte-derived spheres in N2,bFGF+ medium without serum.

### DESCRIPTION OF THE INVENTION

Generally, the present invention provides a stem cell therapy replacement. Isolated pericytes can be used as replacements for stem cells in treating disease.

Recent developments in the ability to isolate, propagate, and differentiate a number of stem cell populations have raised hopes that cell replacement therapies will be effective in the treatment of a large number of disorders. While stem cell research continues to hold great promise, functional application has yet to be realized due in part to restricted availability of large numbers of pluripotent stem cell populations readily available for transplantation. Reliance on embryonic stem cells has obvious limitations. The present invention provides an alternative source of stem cell-like products, the use of pericytes to act as stem cells.

A “pericyte” as used herein is multipotent isolated and purified mesenchymal-like cell and/or pericyte precursors, associated with the walls of small blood vessels. As a relatively undifferentiated cell, it serves to support these vessels, but it can differentiate into a fibroblast, smooth muscle cell, or macrophage as well if required. In order to migrate into the interstitium, the pericyte has to break the barrier, formed by the basement membrane, which can be accomplished by fusion with the membrane. The capillary pericytes include pericytes from skin, muscle, liver, and virtually all organs. Mesenchymal stem cells or MSCs are multipotent stem cells that can differentiate into a variety of cell types. Cell types that MSCs have been shown to differentiate into in vitro or in vivo include osteoblasts, chondrocytes, myocytes, adipocytes, neuronal cells, and, as described lately, into beta-pancreatic islets cells.

The pericytes are administered to the desired areas. While various genetic engineering methods including, but not limited to, transfection, deletion, and the like can be used in order to increase their likelihood of survival or for any other desired purpose, the pericytes of the present invention are able to function effectively without requiring genetic engineering.

The term “stem cell” as used herein is meant to include but is not limited to, a generalized mother cell whose descendants differentiate into various cell types. Stem cells have various origins including, but not limited to, embryo, bone marrow, liver, fat tissue, and other stem cell origins known to those of skill in the art.

Stem cells are capable of self-regeneration when administered to a human subject in vivo, and can become lineage-restricted progenitors, which further differentiate and expand into specific lineages. Further, unless indicated otherwise, the term “stem cells” refers to human marrow stromal cells. Human marrow stromal cells are found in the bone marrow. Bone marrow is soft tissue occupying medullary cavities of long bones, some haversian canals, and spaces between trabeculae of cancellous or spongy bone. Bone marrow is of two types: red, which is found in all bones in early life and in restricted locations in adulthood (i.e., in the spongy bone) and is concerned with the production of blood cells (i.e. hematopoiesis) and hemoglobin (thus, the red color); and yellow, which consists largely of fat cells (thus, the yellow color) and connective tissue.

The terms “stem cell” or “pluripotent” stem cell are used interchangeably to mean a stem cell having (1) the ability to give rise to progeny in all defined hematopoietic lineages, and (2) the capability to fully reconstitute a seriously immunocompromised host in all blood cell types and their progeny, including the pluripotent hematopoietic stem cell, by self-renewal.

The terms “enrich” or “enrichment” as used herein are meant to include, but are not limited to, a process of making rich or richer by the addition or increase of some desirable quality or quantity of substance. In the present invention, enrichment occurs by the addition or increase of more functional cells within or around the tissue being treated. The desired therapeutic effect of the present invention is the ultimate enrichment of functional cells in situ.

The terms “repopulate” or “repopulating” as used herein are meant to include, but are not limited to, the addition or replenishment of cells within or around the tissue being treated. These additionally reinforce the activity of currently functioning cells. Thus, replacement and/or reinforcement of existing cells occurs.

The term “cell therapy” as used herein is meant to include but is not limited to, the administration of pericytes as defined above.

The term “injury” or “disease” as used herein is intended to include, but is not limited to, physical or biological injuries including genetic disorders, diseases, and age onset disorders. The pericytes operate to increase function and/or treat disease by differentiating into functional tissue cells, thereby treating the injury.

The present invention is based on the use of pericyte therapy (similar to stem cell therapy) to treat disease. Although pericytes have different origins, the important common characteristic is that pericytes have the potential to differentiate into various, if not all, cell types of the body based on their microenvironment. Accordingly, in situ location of administered pericytes will the determine the eventual differentiation of the cells.

The pericytes can be administered at the specific location of injury. Alternatively, the pericytes can be placed at general sites within the patient. The pericytes then migrate to site of injury. The administration can be subcutaneously, parenterally including intravenous, intraarterially, intramuscularly, intraperitoneally, and intranasally as well as intrathecally and infusion techniques. The pericytes can be administered by a route that is suitable for the tissue, organ or cells to be treated. They can be administered systemically, i.e., parenterally by intravenous injection, or can be targeted to a particular tissue or organ, such as bone marrow. The pericytes can be administered via subcutaneous implantation of cells or by injection into connective tissue, e.g., muscle.

The dosage of the pericytes varies within wide limits and is Fitted to the individual requirements in each particular case. In general, in the case of parenteral administration, it is customary to administer from about 0.01 to about 5 million cells per kilogram of recipient body weight. The number of cells used depends on the weight and condition of the recipient, the number of or frequency of administrations, and other variables known to those of skill in the art.

The pericytes can be suspended in an appropriate diluent; at a concentration of from about 0.01 to about 5×10⁶ cells/ml. Suitable excipients for injection solutions are those that are biologically and physiologically compatible with the cells and with the recipient, such as buffered saline solution or other suitable excipients. The composition for administration must be formulated, produced and stored according to standard methods complying with proper sterility and stability.

The pericytes can be obtained from a number of different sources. Although the invention is not limited thereto, pericytes can be isolated, purified, and expanded in culture, i.e., in vitro, to obtain sufficient numbers of cells for use in the methods described herein. Unless otherwise stated, genetic manipulations are performed as described in Sambrook and Maniatis, MOLECULAR CLONING: A LABORATORY MANUAL, 2nd Ed.; Cold Spring Harbor Laboratory Press, Cold Spring Harbor, N.Y. (1989).

The pericyte differentiation can be promoted by selectively culturing the pericytes in an enriched environment. The enriched environment preferably contains promoters that specifically promote a specific type of differentiation. The term promoter is intended to include any compound, composition, or other product that can promote the differentiation of pericytes. Examples of such promoters include, but are not limited to, bFGF, SHH, and PDGF. Thus, when a promoter is included in enrichment media in conjunction with pericytes, the pericytes can differentiate into a respective cell type. The promoter functions by signaling to the pericyte the type of cell into which the pericyte should differentiate. For example, it has been determined that capillary pericytes from brain tissue exhibit pluripotent stem cell capability. In the presence of serum, pericytes differentiate along a mesenchymal lineage forming bone, muscle cells, adipocytes, chondrocytes, as well as dendritic cells. In serumless media plus bFGF, pericytes differentiate along a neuronal lineage differentiating into oligodendrocytes, neurons, and astrocytes. Capillary pericytes from tissues of non-CNS origin have similar abilities.

Adult central nervous system (CNS) capillaries contain a distinct population of microvascular cells, the pericyte that are nestin/NG2 positive and in response to basic fibroblast growth factor (bFGF) differentiate into cells of neural lineage. In their microvascular location, pericytes express nestin and NG2 proteoglycan. In serum containing media primary (0 to 7 day old) CNS pericytes are nestin positive, NG2 positive, alpha smooth muscle actin (αSMA) positive, and do not bind the endothelial cell specific griffonia symplicifolia agglutinin (GSA). In serum containing media, pericytes do not undergo neurogenesis but are induced to express αSMA. In bFGF containing media without serum, CNS pericytes form small clusters and multicellular spheres. Differentiated spheres expressed neuronal and glial cell markers. After disruption and serial dilution, differentiated spheres were capable of self-renewal. When differentiated spheres were disrupted and cultured in the presence of serum, multiple adherent cell populations were identified by dual and triple immunocytochemistry. Cells expressing markers characteristic of pericytes, neurons, and glial cells were generated. Many of the cells exhibited dual expression of differentiation markers. With prolonged culture fully differentiated cells of neural lineage were present. Results indicate that adult CNS microvascular pericytes have neural stem cell capability.

More specifically, pericytes can be cultured in serum for up to seven days then transferred to serumless medium+bFGF and retain the ability to differentiate along the neuronal lineage. Capillary pericytes differentiate without previous isolation from their vascular location. When capillaries are cultured in stem cell medium+FGF, pericytes very rapidly form very large differentiated spheres within two to three days at a rate seven to ten times faster and a ten fold higher proliferative response than pericyte replication and differentiation from purified cells. The results show that vascular cells provide trophic support promoting pericyte differentiation. Co-culture of pericyte cells in the presence of capillaries promotes growth. Transplantation of pericytes together with microvessel fragments promotes successful pericyte stem cell replacement in tissues.

Additionally, pericyte differentiation is dependent on initial signaling sonic hedge hog (SHH) for the neuronal lineage and Indian Hedge hog +BMP-2 for mesenchymal differentiation. PDGF selectively promotes oligodendrocyte precursor cell (OPC) formation. Thus, culture conditions can be used to promote differentiation and enrichment of specific cell types.

When capillary spheres are harvested and capillary fragments cultured in the presence of chronic high concentrations of bFGF, tumor-like differentiation and angiogenesis was observed. Tumor formation was associated with adhered vessels on coverslips. Conversion of pericyte stem cells to cancer cells is the result of overexpression or dysregulation of normal signaling mechanisms that result in stem cell differentiation. The system can therefore be used to study of efficacy of anti-tumor medication in vitro. In other words, the pericytes can be cultured and treated with the medication. After treatment the rate of conversion of the pericyte cells to cancer cells can be analyzed such that the higher the conversion rate, the lower the efficacy of the medication.

The following information and the information disclosed in the attached references, which are incorporated by reference in their entireties, describe various methods and materials that can be utilized with the present invention. While specific embodiments are disclosed herein, they are not exhaustive and can include other suitable designs that vary in design and methodologies known to those of skill in the art. Basically, any differing designs, methods, structures, and materials known to those skilled in the art can be utilized without departing from the spirit of the present invention.

### EXAMPLES

### Methods

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al (eds.) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with Flow Cytometry can be used for detection of cells containing specific DNA and mRNA sequences (Testoni et al, 1996, Blood 87:3822.)

### EXAMPLE 1

The CNS microvasculature pericyte has a broad developmental potential. In particular, the neural potential of cells subcultured from isolated rat CNS capillaries was studied. CNS microvessels contain NG2 and nestin-positive pericytes. In serum free medium, pericytes are responsive to bFGF and form clusters of adherent cells and floating spheres composed of cells of multiple neural cell lineage. These cells undergo self-renewal and increase in number after subculturing. By clonal analysis, it was shown that multipotent pericytes differentiate along multiple lineages and may provide trophic support and maintenance in the adult brain.

### Materials and Methods

### Antibodies and Chemicals

Affinity-purified goat anti-mouse and anti-rabbit IgG F(ab)2 fragments conjugated to Red 613 or fluorescein isothiocyanate (FITC) were purchased from CAPPEL (Durham, N.C., USA). Rabbit anti-human Von Willebrand factor (vWF) antibody (IgG) (1:500) was purchased from Dakopatts (Glostrup, Denmark) in either FITC-conjugated or unconjugated form, and mouse anti-human vWF (IgG2) (1:1,000) was purchased from Boehringer Mannheim (Indianapolis, Ind., USA). Rabbit polyclonal anti-glut-1 antibody (IgG) was purchased from Calbiochem (La Jolla, Calif., USA). Mouse monoclonal anti-rat nestin (IgG) was purchased from Chemicon International (Temecula, Calif., USA). Rabbit antibody, directed against rodent GFAP (1:200), and rabbit antibody, directed toward neurofilament 200 (NFL-200) (1:200), were purchased from Sigma (St Louis, Mo., USA). Mouse antibody directed against 5-bromodeoxyuridine (BRDU) was purchased from BD Biosciences (Franklin Lakes, N.J., USA). Cy-conjugated anti-mouse IgG was purchased from Sigma (St Louis, Mo., USA). Mouse anti-NG2 chondroitin sulfate proteoglycan monoclonal antibody (IgG1) and mouse anti rat oligodendrocyte (O4) antigen (1:50) was purchased from Chemicon International (Temecula, Calif., USA).

### Isolation of Microvessels

Rat CNS capillaries were prepared as described previously (Dore-Duffy et al, 1994; Dore-Duffy, 2003). Briefly, brain tissue was removed within minutes of decapitation using sterile technique. Tissue was homogenized in 10 volumes of Dulbecco's modified Eagle's medium (DMEM) using a glass homogenizer and a Teflon® plunger shaved to leave 0.25 mm between the plunger and the glass surface. After 20 up-and-down strokes at 420 r.p.m., the homogenate was centrifuged and the pellet resuspended in 15% dextran in DMEM. The suspension was centrifuged at 5,000 g for 10 minutes (Sorval DuPont, Wilmington, Del., USA), and the pellet resuspended and filtered through 118 mm nylon mesh. The filtrate was passed through an 80 mm nylon mesh. Microvessels (MV) were collected on the 80 mm mesh, washed vigorously from the mesh, and resuspended in DMEM. Microvessel preparations were 80% to 100% capillaries. The MV were greater than 95% viable by trypan blue exclusion.

### Purity of MV

Purity of MV was determined by visual examination and by measurement of g-glutamyltranspeptidase activity using diagnostic kit 545 (Sigma, St Louis, Mo., USA) showing enrichment over starting material. In addition, exclusion of large vessels and the capillary nature of the preparation was confirmed by analysis on the Meridian ACAS 470 laser cytometer with computer-generated size determinations (Dore-Duffy et al, 1994). Staining of microvessel capillary preparations indicated that there were no neurons or glial cell contaminants (Dore-Duffy et al, 1994; Dore-Duffy, 2003).

### Primary Pericyte Cultures

Capillaries were prepared as detailed above and suspended in DMEM (Sigma, St Louis, Mo., USA) supplemented with 50 mg/mL penstrep, 2.5 mg/mL nystatin and 0.1% collagenase type II (Worthington corp., Freehold, N.J., USA) and incubated at 37° C. overnight in a shaker bath. After incubation, disrupted MV fragments and single cells were vigorously pipetted to disrupt any remaining fragments then centrifuged. Cells were pelleted and resuspended in DMEM plus 2% fetal calf serum (FCS) and 1% antibiotics and negatively purified by fluorescence-activated cell sorting (FACS) (Balabanov et al, 1996; Dore-Duffy, 2003). To confirm purity, cells (10⁵/mL/plate) were allowed to adhere for 4 hours on coverslips then stained for EC-specific and pericyte markers. Cells were plated on uncoated plastic and then nonadhered cells vigorously washed off. Nonadhered cells were again washed off after 24 hours. Pericytes began to proliferate by 24 to 48 hours and became confluent by days 7 to 8 depending on the plating density. Purity of pericyte cultures was assessed after dual staining of cultured FACS sorted cells for griffonia symplicifolia agglutinin (GSA)-FITC binding or factor VIII and for alpha smooth muscle actin (αSMA) after 3 days in culture (Herman and D'Amore, 1985). FACS sorted GSA cultures were 100% pericytes (GSA-αSMA+) (Dore-Duffy, 2003). GSA+ cell populations contained roughly 0% to 3% αSMA+ cells. No GFAP+, NF1+ or O4+ cells were observed.

### Primary EC Cultures

Microvessels were digested with 0.1% collagenase for 4 to 6 hours at 37° C. MV were vigorously pipetted to produce tiny fragments. Approximately 10⁴ MV fragments per plate were allowed to adhere overnight on collagen-coated Petri dishes. Nonadhered fragments and loosely adhered cells were vigorously shaken and washed off after 24 hours. Using this technique, cultures were routinely 90% to 96% EC. Total removal of pericytes required flow cytometric removal of freshly isolated CD11b-positive cells (Balabanov et al, 1996). Even with flow cytometry it was almost impossible to completely eliminate pericytes as GSA+ cell populations usually have small percentages of αSMA+ cells after 3 days in culture.

Culture of bFGF-Responsive Cells

The viability of cells was determined by trypan blue exclusion. Viable cells (200/cm²) were plated in 35 mm Petri dishes in DMEMF-12 with N2 supplement plus 20 ng/mL human recombinant bFGF (medium described below) (Gibco, Grand Island, N.Y., USA). Fresh medium was carefully added every 2 to 3 days after centrifugation of the plates at 800 g for 10 minutes. The number of free-floating spheres was counted after 20 to 25 days in culture. Some spheres were cultured on poly-L-ornithine (Poly-O)-coated glass coverslips for immunocytochemistry. Other spheres were used in experiments to establish self-renewal and in subcloning experiments. Pericyte spheres were transferred to conical test tubes in bFGF supplemented medium and diluted to yield one sphere/tube. Spheres were mechanically disassociated by vigorous up and down pipetting. One cell was added per microwell using a limiting dilution technique. Cells were incubated in bFGF containing medium. Approximately 10% of cells survived and produced new spheres. Spheres were transferred to Poly-O-coated slides. A second subcloning step was used to make a third group of spheres.

### Immunocytochemistry

Pericyte spheres were grown on Poly-O-coated coverslips for 5 days in bFGF containing medium then transferred to medium with no growth factors. Coverslips were fixed for 30 minutes at room temperature in 4% paraformaldehyde then washed 3 times in phosphate-buffered saline (PBS). Coverslips were treated 10 minutes with 0.01% Triton X-100 in the presence of blocking antibody. After washing 3 times in PBS, the coverslips were MV and primary cultured EC and pericytes were allowed to dry on alcohol washed glass coverslips or poly-O and fixed with 3% paraformaldehyde. Coverslips were washed in PBS with 1% bovine serum albumin, permeabilized with Triton X-100 and then stained with antibody directed against indicated markers, or with control antibodies. All primary antibodies were used at antibody excess and secondary antibodies at predetermined saturation density (1:100 dilutions for nestin, GFAP and 1:200 dilution for antibodies directed against CD11b, NF1, and vWF). Coverslips were incubated 30 minutes at room temperature, washed 3 times then incubated with fluorochrome-conjugated secondary antibody (affinity-purified anti-IgG F(ab)0 2 fragments 1:100 dilution) as appropriate. A second antibody alone and/or isotype control antibody was used as a control. In dual label experiments, species similar immunoglobulins were not used. Immunofluorescence was examined with a fluorescence microscope Orthoplan-2 (Zeiss, Germany) using ×25 and ×60 oil objectives. Percentage of positive MV or cells was calculated after counting a minimum of 300 cells or MV. Coverslips were photographed with a 35 mm CONTAX 165 MT camera (Kyocera Corp., Japan).

### Fluorescence-Activated Cell Analysis/Sorting (Dore-Duffy, 2003)

After enzyme treatment, single cell suspensions were incubate with EC binding FITC-conjugated GSA 1:100 for 60 minutes in the cold. GSA-FITC-positive cell populations were removed. For analysis, suspensions were dual labeled with anti-rat nestin antibody and GSA-FITC. Cy-conjugated anti mouse IgG was used as second antibody. Cell suspensions were fixed then analyzed by a Becton-Dickerson fluorescence activated cell sorter. Isotype similar nonspecific antibody was used as controls and values for percentage subtracted.

### Polymerase Chain Reaction Techniques

Reverse transcriptase polymerase chain reaction (RT-PCR) was performed using total RNA from rat brain (Mullis and Faloona, 1987). Synthesis of cDNA was performed as previously described (Mullis and Faloona, 1987). Two microliters of cDNA per assay point was amplified using forward and reverse gene-specific primers listed in Table 1. PCR products were isolated on 1.5% agarose gels. Quantitative (real-time) PCR (Kwok and Higuchi, 1989; Faloona et al, 2005) was performed on selected samples as indicated in the text. Experiments were performed in the Wayne State University DNA facility using Applied Biosystems Inc. Prism 7700 sequence Detection System (Foster City, Calif., USA). Parameters include 10 minutes, 95° C., 40 cycles of 30 seconds at 95° C., 2 minutes at 60° C. using 2 mL cDNA, 400 nmol/L target gene primers and 12.5 mL of SYBR GREEN 1 dye. An internal reference dye (Rox) was used to normalize for non-PCR related fluctuations in fluorescence that may occur from well to well.

### Results

### Purity of Microvessel and Primary Pericyte Cultures

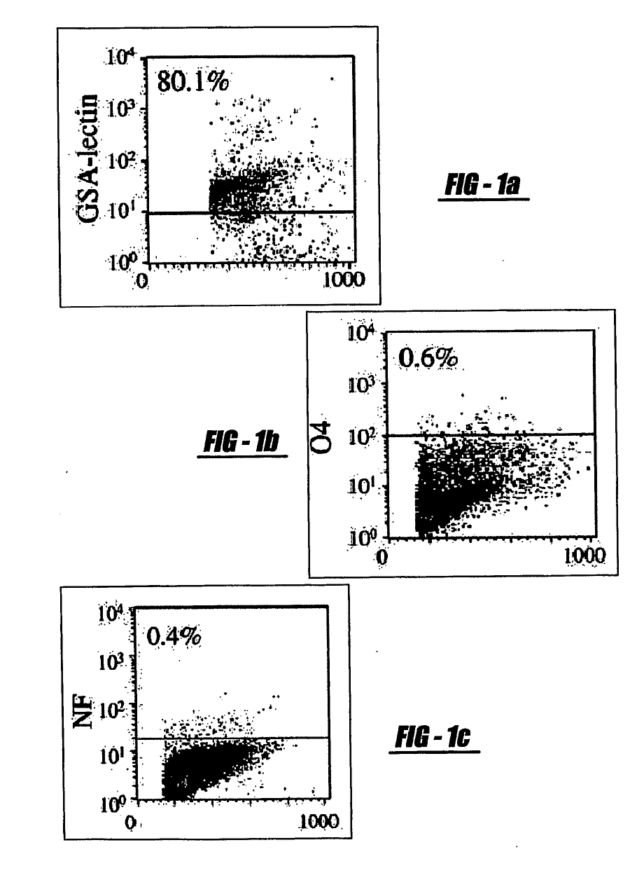

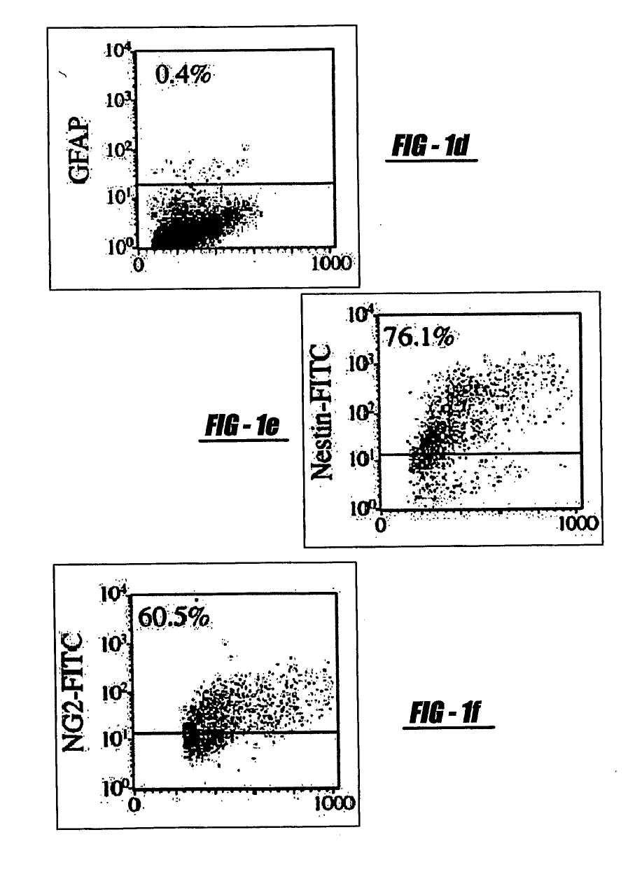

Central nervous system capillary preparations were isolated and enzymatically disrupted as detailed above. If capillaries are carefully isolated they can be prepared with no cellular contamination. However, if isolation techniques are not performed carefully or performed incorrectly then cellular contaminants can include neurons. Poorly controlled preparations can also include astrocytes as well as glial cells. While the laboratory routinely isolates pure capillary preparations for pericyte subculture, care was taken to document purity to prove the validity of subsequent data. Capillary digests were examined for purity using immunocytochemistry and then analyzed by FACS analysis (FIG. 1). Capillary cell preparations did not contain O4-positive cells, GFAP-positive cells, or neurofilament antigen-positive cells. Capillaries were positive for GSA lectin-binding cells (ECs) and GSA lectin-negative/NG2-positive cells (pericytes) (Balabanov et al, 1996; Dore-Duffy, 2003; Balabanov and Dore-Duffy, 1998). Muscle-specific actin (αSMA), although an in vitro marker for pericytes, is not expressed on 100% of pericytes in vivo. Thus, a SMA was not used to identify pericytes in stained capillaries. Using the above FACS criteria as well as additional tests performed for capillary purity, it was concluded that the capillary preparations were pure and devoid of glial and neuronal cell contamination.

More specifically, FIGS. 1A through 1F show the purity of freshly isolated CNS capillary suspensions. Freshly isolated CNS capillary suspensions were enzymatically disrupted (Dore-Duffy, 2003). After disruption, single-cell suspensions were stained using immunocytochemical techniques and the antigenic properties of cell populations analyzed by flow cytometry. CNS capillary digests contained cells that bound FITC-GSA lectin (ECs) and stained positively for the expression of vWF. Preparations also contained nestin-positive cells and NG2-positive cells. Capillary preparations did not contain O4+ cell populations, cells that stained positive for expression of neurofilament (NF), or cells that expressed GFAP. Cultured FACS sorted GSA cells expressed αSMA.

### Capillaries and Primary Pericytes Express Nestin/NG2

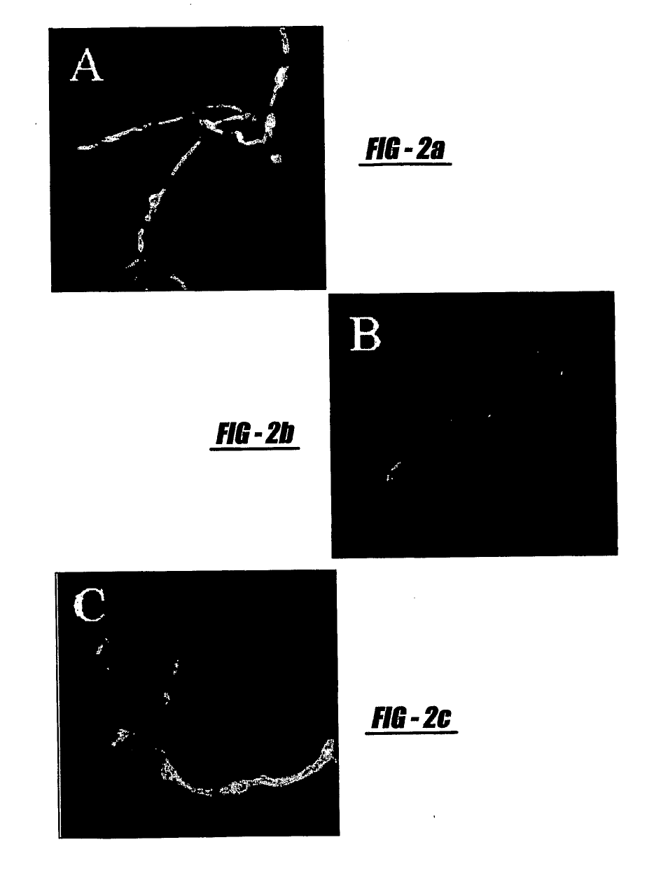

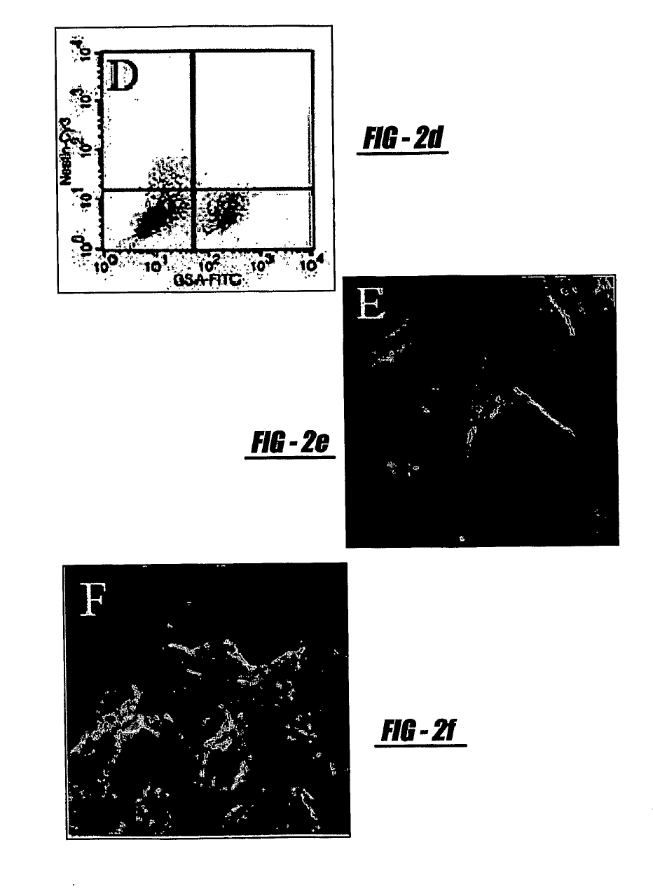

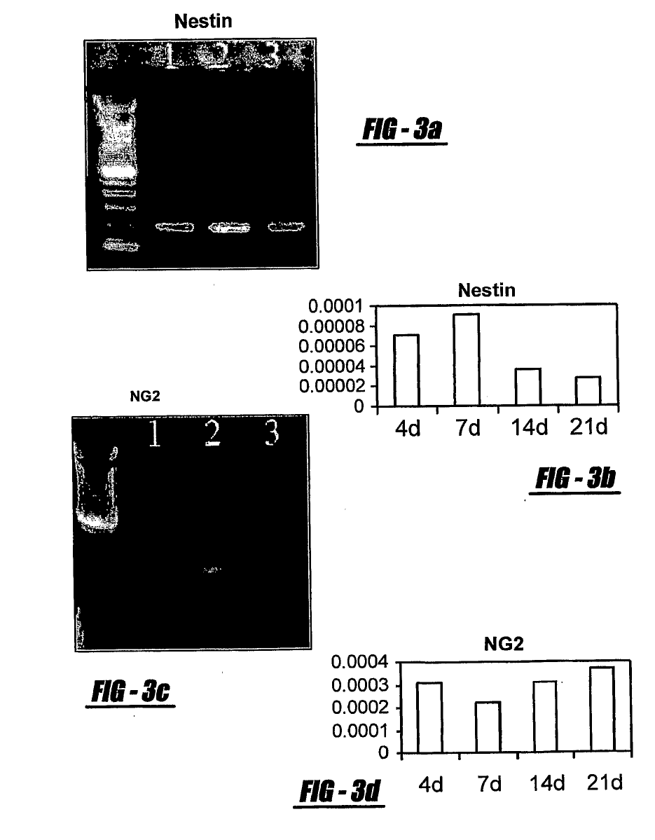

Pure capillary preparations were prepared as detailed above and subcultured to produce primary pericytes as detailed in Methods. Capillaries were dual stained for expression of EC-specific markers (vWF; GSA lectin binding activity) and nestin. Capillaries were also dual stained for expression of NG2/nestin. Primary pericytes (7-day-old) were dual stained for expression of αSMA/nestin, and NG2/nestin. 4,6-diamidino-2-phenylindole (DAPI) was used to stain nuclei. Results are shown in FIG. 2. Capillaries expressed Von Willebrand factor (vWF) and nestin (FIG. 2A). Von Willebrand factor staining is distributed throughout the vessel, whereas nestin staining appeared to concentrate near round versus elongated nuclei. The pattern of nestin staining within the vessel is consistent with localization of antigen in pericytes (Balabanov et al, 1996). There was no evidence of co-localization of nestin with vWF. Capillary preparations also stained positively for NG2/nestin (FIG. 2B) and vWF/NG2 (FIG. 2C). To confirm that microvascular capillaries contain a nestin-positive cell population, a second set of experiments were performed. Freshly isolated capillaries were enzymatically disrupted and single cells resulting from these digests were dual stained with FITC-conjugated GSA lectin and antibody directed against nestin. Dual stained cell suspensions were examined using FACS analysis (FIG. 2D). Nestin expression was localized in GSA cell populations (FIG. 2D) consistent with localization within pericytes (Dore-Duffy, 2003). When GSA/nestin+ cells were plated, 100% became αSMA+ by 4 to 7 days in culture. These cells expressed immunologically reactive αSMA/nestin and αSMA/NG2 as shown in dual stains (FIGS. 2E and 2F). Capillaries and primary pericyte cultures also expressed nestin and NG2 transcripts by RT-PCR (FIGS. 3A and 3B). Taken together, the results indicated that CNS capillary pericytes express nestin and NG2 in situ and in primary culture. Pericyte expression of nestin transcripts decreased with time in culture when assayed by quantitative real-time PCR (FIG. 3C). However, NG2 mRNA, transcripts were expressed up to 3 weeks (FIG. 3D).

FIG. 2 depicts the characterization of nestin/NG2-positive cells in CNS capillaries. Freshly isolated CNS capillaries were dual stained for expression of vWF(green)/nestin(red) (FIG. 2A); NG2(red)/nestin(green) (FIG. 2B); and NG2(red)/vWF(green) (FIG. 2C). Nuclei were stained with DAPI. Freshly isolated CNS capillaries were enzymatically digested and cell suspensions stained for expression of nestin and the ability to bind FITC-conjugated GSA lectin and analyzed by flow cytometry (FIG. 2D). Enzymatically disrupted cell suspensions were also used to culture primary pericytes (Dore-Duffy, 2003). Primary pericytes were dual stained for expression of αSMA(red)/nestin(green) (FIG. 2E) and αSMA(red)/NG2(green) (FIG. 2F).

FIGS. 3A through 3D depict the expression of nestin and NG2 transcripts in cultured CNS primary pericytes. Freshly isolated CNS capillaries were enzymatically digested and pericytes subcultured. Primary pericytes were cultured in DMEM plus FCS and in N2bFGF medium. Total RNA was isolated from capillaries pericytes and from pericyte derived spheres. Reverse transcriptase polymerase chain reaction analysis indicated that capillaries (lane 1), pericytes (lane 2), and spheres (lane 3) expressed mRNA transcripts for nestin (FIG. 3A) and NG2 (FIG. 3B). Primary pericytes were also cultured for varying periods of time up to 21 days in DMEM plus serum. Nestin and NG2 transcripts were analyzed by quantitative real-time PCR (FIGS. 3C and 3D). Results indicated that pericyte expression of nestin decreased with time in culture (FIG. 3C) while NG2 mRNA expression remained relatively stable (FIG. 3D) during the culture period.

### Primary Central Nervous System Microvascular Pericytes Proliferate in Response to Basic Fibroblast Growth Factor

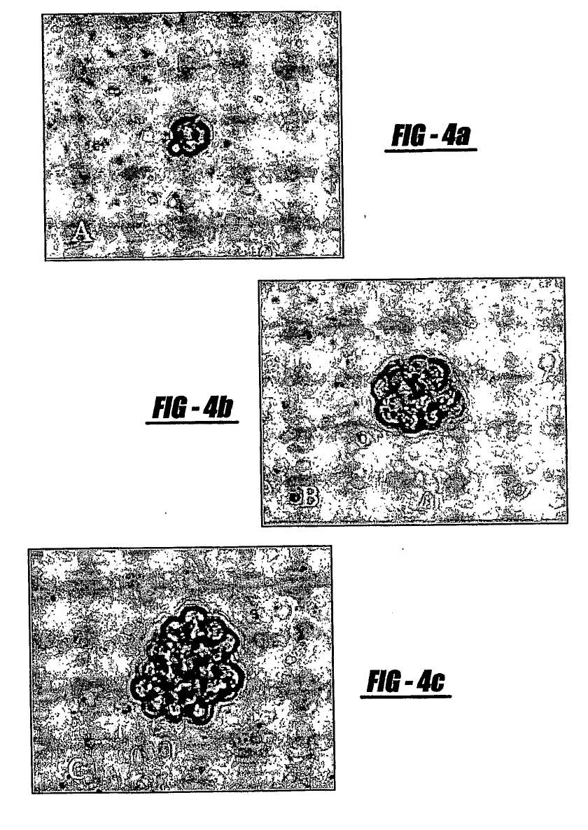

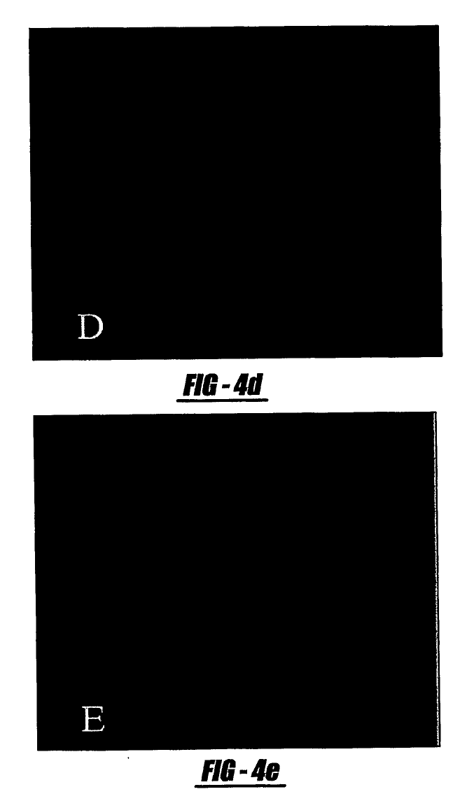

Primary pericytes were plated at a low density (200 cells/cm²) in normal culture medium, and in DMEM F12 (no serum) with N2 supplement with and without bFGF (20 ng/mL). In normal culture medium (DMEM+20% FCS), pericytes display typical morphology. On plastic, pericytes appear large with multiple short processes (Balabanov et al, 1996, 1999; Balabanov and Dore-Duffy, 1998). However, in N2 medium supplemented with bFGF, pericytes displayed altered culture morphology (FIGS. 4A through 4E). Few cells adhered to the culture dish and approximately ¼ of the cells died. Trypan blue-positive cells appeared by 2 days. Adhered as well as nonadhered cells formed small spherical clusters that, with continued growth, lifted from the culture dish. The majority of growing spheres remained in suspension growing in size as shown in FIGS. 4A-4C. Pericyte spheres were nestin+ (FIG. 4D) and labeled with BRDU (FIG. 4E). Pericyte-derived spheres expressed nestin and NG2 transcripts (FIGS. 3A and 3B, lane 3).

More specifically, FIGS. 4A through 4E show that the primary CNS pericytes are responsive to bFGF. Primary CNS pericytes were cultured in medium with N2 supplement and bFGF for variable periods of time. The majority of cells remained in suspension although a few adhered to the culture dish. Both adhered and floating cells replicated forming spheres that increased in size (FIG. 4A through FIG. 4C). Cells at two to four days in culture are shown in FIG. 4A, 1 week in FIG. 4B and 2- to 3-week-old spheres in FIG. 4C. Three-week-old spheres were nestin+ (FIG. 4D) and labeled with BRDU (FIG. 4E). Nuclei were stained with DAPI in both FIG. 4E and FIG. 4D.

### Basic Fibroblast Growth Factor-Responsive Central Nervous System Pericytes are Multipotent and Differentiate

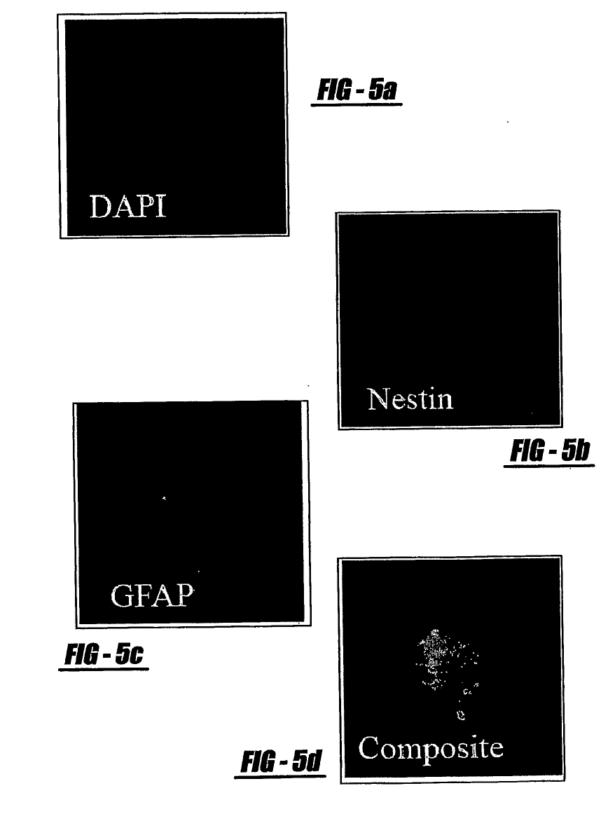

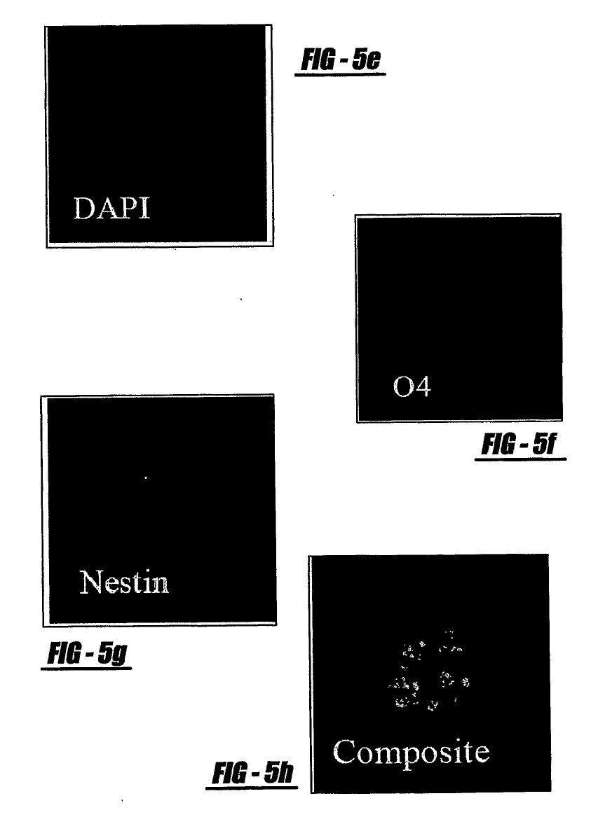

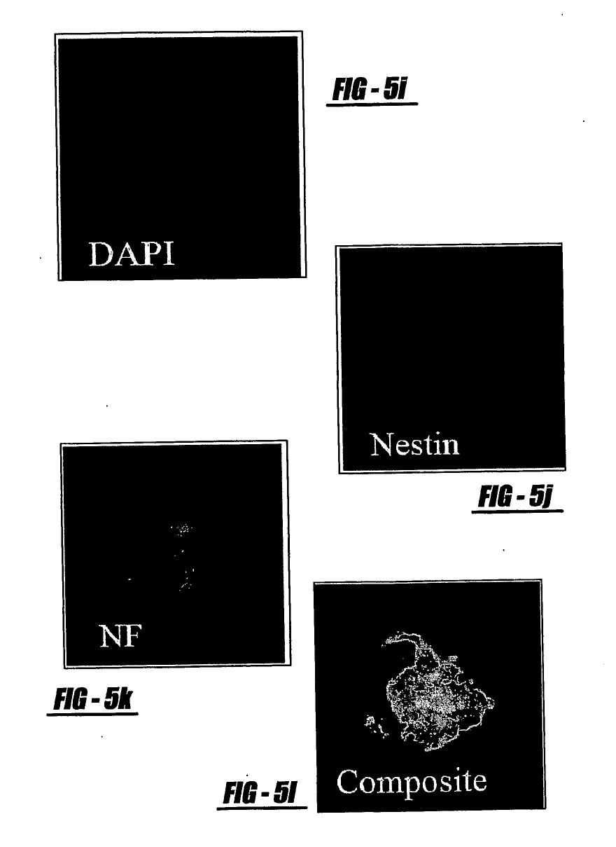

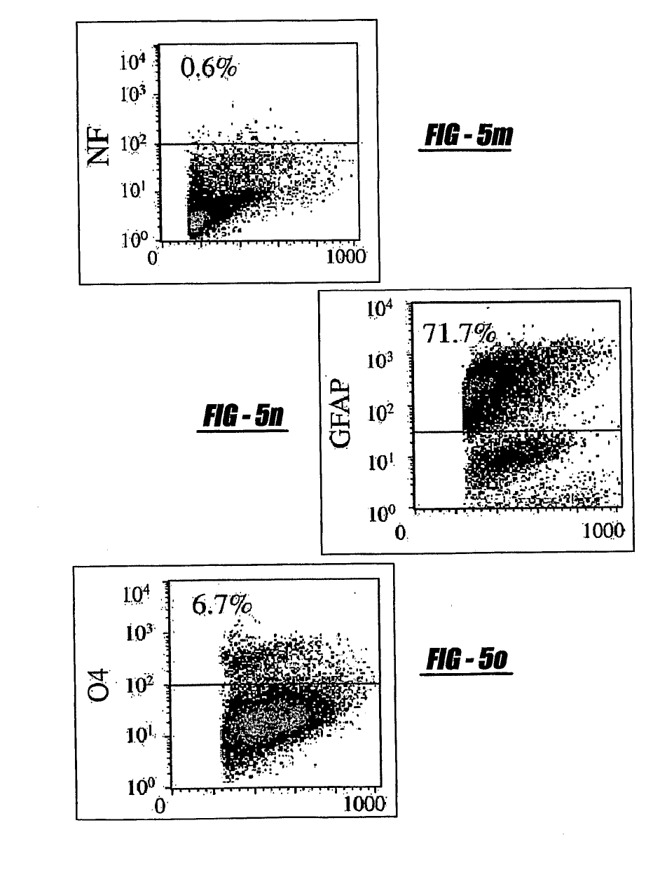

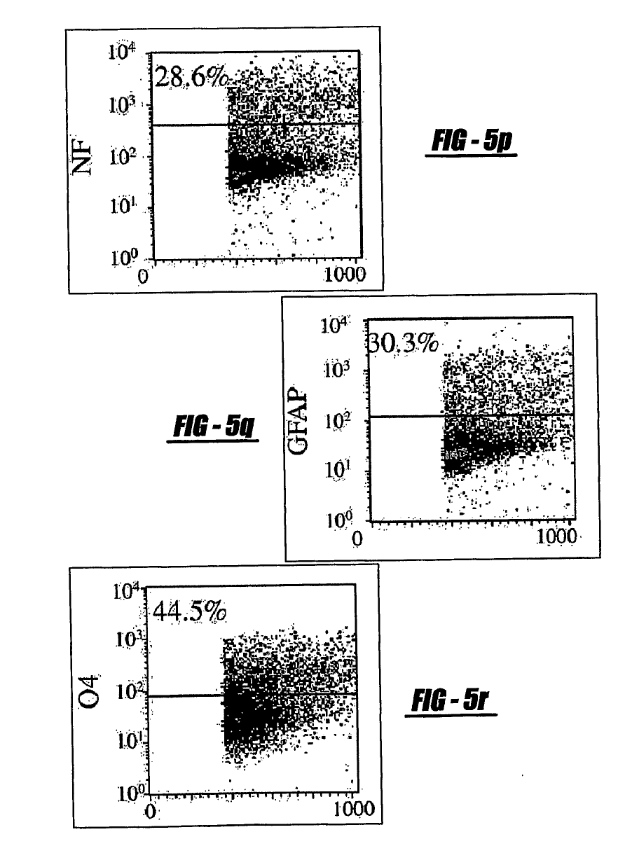

Whether bFGF-responsive CNS pericyte-derived spheres could differentiate into the major cell populations present in the CNS was investigated. Individual 20 to 25 day old spheres were transferred to glass coverslips coated with poly-O. Coverslips were prepared for immunocytochemistry and stained for astroglial, oligodendroglial, neuronal markers and the chondroitin proteoglycan NG2. Results indicate that each primary BRDU+ sphere differentiated into multiple cell types (FIG. 5A through FIG. 5L). Astroglial cells were identified by expression of GFAP, oligodendrocyte precursors by O4-positive staining, and neuronal differentiation by expression of neurofilament (FIG. 5A through FIG. 5L). All spheres were nestin positive. All spheres also expressed NG2. Differentiation of pericyte spheres into cells of neurolineage was confirmed by FACS analysis (FIG. 5M through FIG. 5R) and by analysis of specific transcripts by quantitative real-time RT-PCR.

FIG. 5A through FIG. 5R show the characterization of differentiated pericyte-derived spheres. Two- to three-week-old pericyte-derived spheres were characterized by immunocytochemistry (FIG. 5A through FIG. 5L) and by flow cytometry (FIG. 5M through FIG. 5R). Pericyte-derived spheres contained cells that stained positively for dual expression of nestin/GFAP (FIG. 5A through FIG. 5D); nestin/O4 (FIG. 5E through FIG. 5H) and nestin/NF (FIG. 5I through FIG. 5L). Pericyte-derived spheres were disrupted, stained and analyzed by flow cytometry (FIG. 5P through FIG. 5R). Primary rat astrocyte preparations were similarly analyzed as controls (FIG. 5M through FIG. 5O). FACS analysis indicated that spheres showed evidence of differentiation to the neural lineage. Pericyte derived spheres contained cells that expressed GFAP, NF and O4+. Astrocyte control cell suspensions expressed GFAP, a small percentage of O4 positive cells, and no cells expressing NF.

### Self-Renewal of Multipotent Basic Fibroblast Growth Factor-Responsive Central Nervous System Pericytes

To show that self-renewal, bFGF-responsive pericyte-derived spheres were serially subcloned. Individual cells from the primary isolation produced spheres (Table 2). When these cells were disrupted, serially diluted, and plated at low density in the presence of bFGF, new secondary spheres and a few small adherent clusters were formed. A second generation as well as a third generation of spheres could be produced. Secondary and tertiary spheres were nestin positive and displayed evidence of differentiation (Table 2). The percentage of differentiated spheres increased somewhat with serial subcloning.

### Differentiated Spheres Returned to Serum-Containing Medium

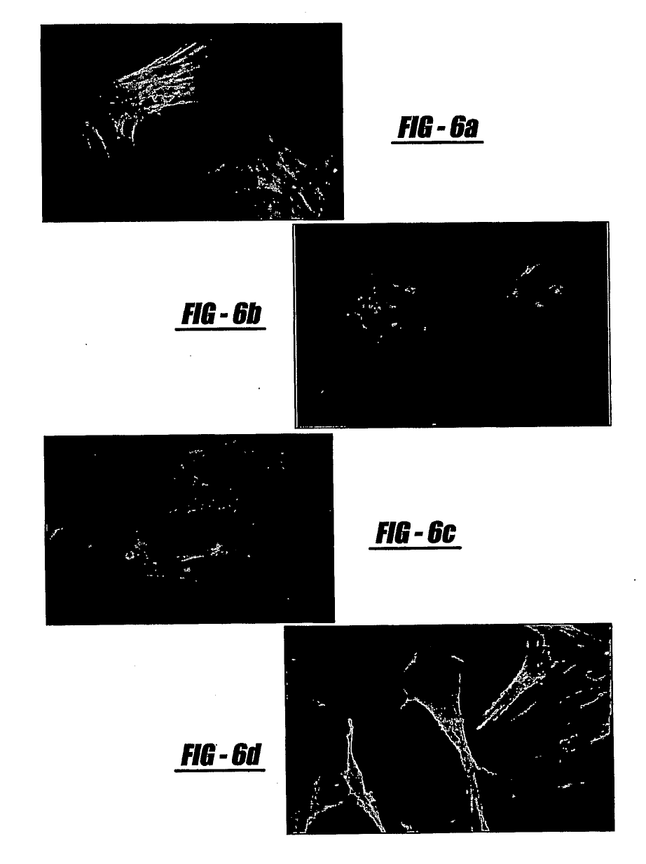

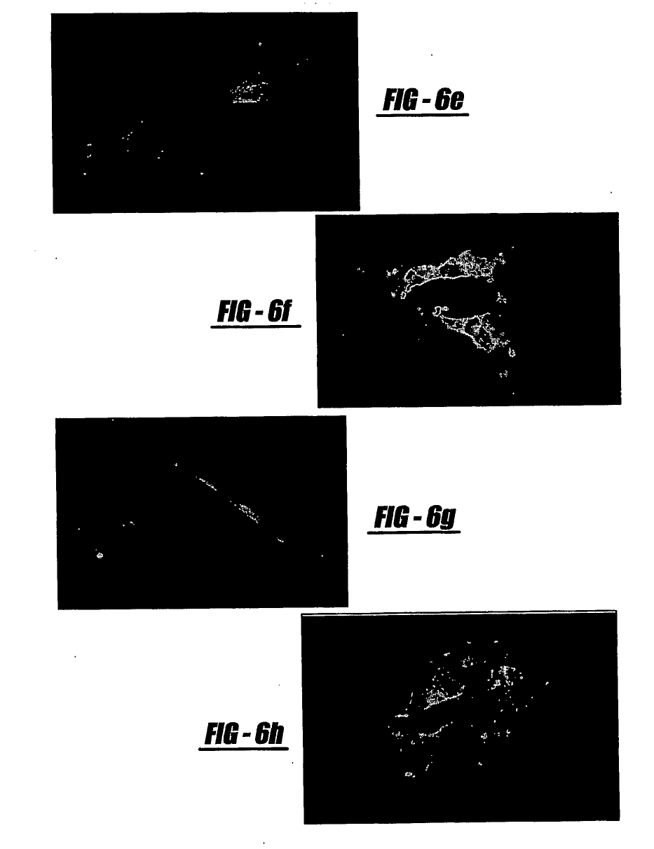

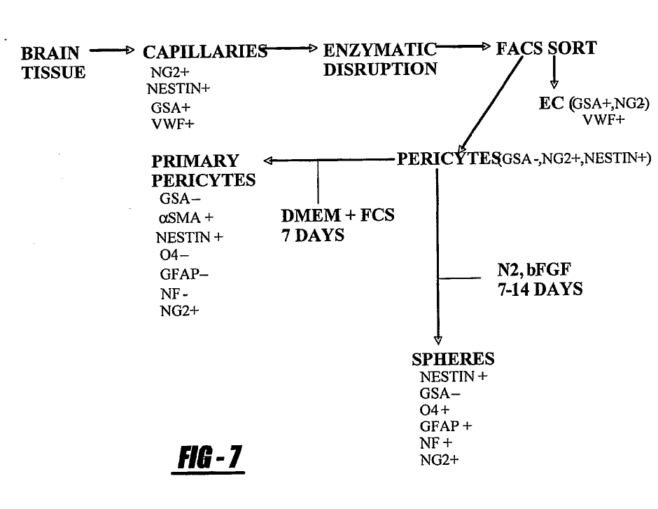

Primary and secondary spheres were disrupted and returned to DMEM supplemented with serum and allowed to adhere to coverslips for 4 to 7 days (FIG. 6A through FIG. 6H). Coverslips were prepared for immunocytochemistry to document the presence of differentiated cells of neural lineage. Double and triple labeling techniques revealed that each sphere contained cells that, after plating in serum-containing medium, continued to exhibit neuronal, astroglial, and oligodendroglial markers (FIG. 6A through FIG. 6H). Each sphere contained cells that were αSMA/NG2 positive and nestin/αSMA positive and displayed a morphological appearance consistent with pericytes (FIG. 6A and FIG. 6B). Many αSMA+ cells coexpressed GFAP, O4, or NF (FIG. 6C through FIG. 6E). A number of nestin-positive cells that exhibited dual expression of neuronal and glial markers were observed, as well as cells that expressed NF and morphologically resembled neurons (FIG. 2B). Dual expression of O4/GFAP, NF/O4, and GFAP/NF were observed (FIG. 6F through FIG. 6H). The percentage of each cell population was determined at 5 days of culture and is shown in Table 3. The majority of cells exhibited pericyte morphology (30% NG2). Dual expression of αSMA and GFAP was seen in approximately 9% of cells. Coexpression of αSMA and O4 was observed in 6% of cells. Differentiated O4-positive cells represented approximately 6% of the population. αSMA/NF dual labeled cells compose approximately 11% and total NF+ cells equal 12%. Cells dual labeled for GFAP and αSMA represent 9%. GFAP+ cells with morphology characteristic of astrocytes represented only 3% or less. A proportion of cells were also αSMA+, nestin+, NF+ (3%). GFAP+/O4+ cells (5%) were observed, as were NF/O4+ cells (7%) and GFAP/NF+ cells (8%).

FIG. 6A through FIG. 6H depict the antigenic properties of cells cultured from pericyte derived spheres. CNS primary pericyte-derived spheres (21 days old) were disrupted and cultured for 5 to 7 days in DMEM supplemented with 20% serum. Cells were dual stained for expression of indicated markers using immunocytochemistry. Nuclei were stained with DAPI. Differentiated spheres yielded cells that exhibited expression of: αSMA(red)/nestin(green); αSMA(red)/NF(green), NF(green); αSMA(red)/GFAP(green); and αSMA(red)/04(green) (FIG. 6A through FIG. 6D). Spheres also generated αSMA(red)/NG2(green) positive cells (FIG. 6E) that were also nestin positive. The spheres generated cells that exhibited dual expression of GFAP(green)/04(red), 04(green)/NF(red) and GFAP(green)/NF(red) (FIG. 6E to FIG. 6H). Spheres also generated cells that expressed only NF (FIG. 6B) seen by 4 days in culture.

CNS pericytes cultured for prolonged periods in the presence of serum exhibit multipotentiality and differentiate to a macrophage-like phenotype then, with a prolonged culture period, differentiate to an osteogenic phenotype. CNS pericytes form nodules that express bone-specific protein by 18-21 days culture. Similar results have been reported for retinal pericytes (Reilly et al, 1998).

Adult multipotential stem cells have been described in tissue from brain (Mckay, 1997; Temple, 2001; Sprading et al, 2001), bone marrow (Terskikh et al, 2001), skin (Goodyer et al, 2001), skeletal muscle (Peng and Huard, 2004), and periostium (Simon et al, 2003). While the concept of a vascular stem cell has been discussed, much of this literature has focused on new vessel formation during angiogenesis in non-CNS tissue, as well as the possible stem cell nature of the EC (Reilly et al, 1998; Lutton et al, 2002). Nestin-positive cells within microvascular capillaries are GSA/vWF, and, on culture in serum containing medium, form αSMA+ cells. However, co-culture of EC and pericytes enhanced the number of differentiating primary spheres. Most fully differentiated αSMA+ pericytes are not multipotent and do not form neurospheres when removed from serum-containing medium and cultured in bFGF-containing N2 medium without serum. Pericytes at this stage of differentiation can be committed to the mesenchymal lineage.

Neurogenesis occurs in foci (niches) in close association with microvessels (Doetsch, 2003; Palmer et al, 2000). These multicellular regulatory niches promote self-renewing cell populations. The cellular constituents of the microvasculature are important in regulation of differentiation. The microvasculature is a source of adult stem cells and that these cells are the pericytes. That pericytes are found in most tissue may help explain the transdifferentiation of adult stem cells from one tissue type to another.

Adult stem cell proliferation and differentiation is induced in response to environmental stress (Zhu et al, 2005). Proliferating stem cells are often found in a perivascular location (Song et al, 2002). However, the exact mechanism that regulates induction and proliferation, as well as the role of the stem cell in adaptation to stress injury is not known. Pericytes migrate from their vascular location in response to stress injury (Dore-Duffy et al, 2000) and remain in a perivascular location where they proliferate during the angiogenic response (Gerhardt and Betsholtz, 2003). Pericytes regulate angiogenesis (Balabanov and Dore-Duffy, 1998; Gerhardt and Betsholtz, 2003; Otani et al, 2000; Nehls et al, 1992; Hirschi and D'Amore, 1997; Yonekura et al, 1999) during: (1) initiation (Otani et al, 2000); (2) sprout formation and migration (Nehls et al, 1992); and (3) maturation and termination (Gerhardt and Betsholtz, 2003; Hirschi and D'Amore, 1997; Yonekura et al, 1999). Pericyte signaling molecules that are involved in regulation of angiogenesis are also involved in neurogenesis (Jin et al, 2002; Louissant et al, 2002). Pericytes synthesize the proangiogenic cytokine vascular endothelial cell growth factor (VEGF) (Yonekura et al, 1999). VEGF augments periycte proliferation in an autocrine fashion (Yonekura et al, 1999) and promotes differentiation of multipotent chondrocytic stem cells (de la Fuente et al, 2004). Pericytes are also responsive to growth factors and other signaling molecules important in regulation of neurogenesis (Nico et al, 2004; Balabanov and Dore-Duffy, 1998; Yonekura et al, 1999). Thus, the response to injury and stress at the tissue level is co-regulated with neurogenesis in the adult and tied into regulation of the cell cycle. Regulation of the cell cycle is important to cell repair and survival and is an integral component of the stress response. The arresting of G1-S transition involves a complex interaction of cullins, p53-responsive proteins, glucose response proteins, cyclins and other regulatory elements that render the cell competent to proliferate after reception of a subsequent signal (Sciandra et al, 1984; Amellelum and Peterson, 1991; Little et al, 1994; Jackson, 1996). A similar complex sequence of regulatory factors and cell cycle regulation is important in differentiation during neurogenesis and is involved in mobilization of adult stem cell activity.

Pericytes synthesize heparin sulfate proteoglycans as well as other matrix proteins (Dienfenderfer and Brighton, 2000; Ozerdem et al, 2002) and are thought to be responsible for the laying of the basal lamina during development (Balabanov and Dore-Duffy, 1998). CNS pericytes are NG2+ confirming previous observations in other tissue (Ozerdem et al, 2001). Adult neurogenesis is thought to be due to an NG2+ progenitor cell that is intrinsically multipotent (Chekenya et al, 2002; French-Constant and Raff, 1986; Wolswijk and Noble, 1998; Belachew et al, 2003; Aguirre et al, 2004). Heparin sulfate proteoglycans tether and present many factors involved in adult neurogenesis (Hienola et al, 2004). Pericyte synthesis of extracellular matrix proteins can therefore have profound effects on the presentation of regulatory molecules within the stem cell niche and shows that pericytes have an important role in homeostasis as well as neurogenesis.

CNS microvascular pericytes are nestin/NG2 positive and are a source of adult multipotential progenitor cells. The CNS pericyte is a source of purified viable multipotent stem cells with the potential of directed neurogenesis and is important to future therapeutic strategies.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention can be practiced otherwise than as specifically described.

TABLE 1
Primers used for RT-PCR and real-time PCR

| | | |
|---|---|---|
| Nucleotide | Up | Down |

| | | |
|---|---|---|
| Rat GFAP | 5-GAA GAA AAC CGC ATC ACC AT-3 | 5-GGC ACA CCT CAC ATC ACA TC-3 |
| Rat Nestin | 5-CGC CGC TAC TTC TTT TCA AC-3 | 5-GCA GCT GGT TTT GCT CTT CT-3 |
| Rat-NG2 | 5-ATG CCC ACT GTA GCC AAA AG-3 | 5-GTG TCA CCA GCT AGG CCA TT-3 |
| Rat NF-H | 5-CTC TCA GAG GCA GCC AAA GT-3 | 5-TGC TGA ATT GCA TCC TGG TA-3 |

TABLE 2
Self-renewal properties of pericyte bFGF-responsive cells

| Primary | No. of | No. of | No. of tertiary | |
|---|---|---|---|---|
| spheres tested | secondary | differentiated | differentiated | No. of |
| (N = 3) | spheres^{a} | spheres^{b} | spheres^{c} | spheres^{d} |
| 25 | 56 + 12 | 33 + 7 (67) | 72 + 21 | 59 + 10 (82) |

^{a}Serial diluted individual primary spheres were dissociated to single cells and introduced to bFGF containing medium at clonal density. Cells were incubated 21 days.
^{b}Number of secondary spheres containing differentiated GFAP+, 04+, or NeuN+ cells. Percentage values in parentheses.
^{c}Secondary spheres were dissociated and introduced to bFGF containing medium for 21 days.
^{d}Number of tertiary spheres with differentiated cells. Percentage values in parentheses.

TABLE 3
Cultures from differentiated spheres^{a}

| Antigens^{b} | Percentage^{c} |
|---|---|
| Nestin/αSMA/NG2 | 30 |
| αSMA/GFAP | 9 |
| αSMA/O4 | 4 |
| αSMA/NF | 11 |
| O4/GFAP | 5 |
| O4/NF | 7 |
| NF/GFAP | 8 |
| NF | 12 |
| GFAP | 3 |
| O4 | 6 |

^{a}Secondary spheres were disrupted and plated in DMEM plus 29% serum for 4-5 days.
^{b}Cells were stained for expression of antigen (as detailed above).
^{c}The percentage of positively stained cells was determined using a fluorescence microscope. A minimum of 20 cells per coverslip and three coverslips per determination were counted.

What is claimed is:

1. A method of promoting perictye differentiation by selectively culturing pericytes in an enriched environment containing a promoter specific to a type of differentiation.

2. The method according to claim 1, wherein said culturing step includes signaling differentiation of the pericyte more specific to the modified enriched environment.

3. The method according to claim 2, wherein said signaling step includes signaling differentiation of the pericyte more specific to the modified environment enriched for mesenchymal differentiation.

4. The method according to claim 2, wherein said signaling step includes signaling differentiation of the pericyte more specific to the modified environment enriched for oligodendrocyte precursor cell formation.

5. Isolated and purified multipotent pericytes or pericyte precursors.

6. The use of the pericytes of claim 5 in treating disease.

7. The pericytes of claim 5 for use as replacements of cells in treating disease.

8. A stem cell therapy replacement comprising isolated and purified multipotent pericytes or pericyte precursors.

9. A treatment of disease comprising an effective amount of isolated and purified multipotent pericytes or pericyte precursors.

10. The treatment according to claim 9, wherein said pericytes have multipotentiality.

11. A screen for testing efficacy of anti-tumor medication, said screen comprising isolated, multipotent pericytes and an anti-tumor medication.
